(19) United States
(12) Patent Application Publication
(10) Pub. No.: US 2003/0190254 A1
(43) Pub. Date: Oct. 9, 2003

(54) Method for ultra-violet disinfecting of compressed air

(76) Inventor: Frank Falat, Berea, OH (US)

Correspondence Address: FRANK FALAT, 336 DAISY AVE., BEREA, OH, 44017, US

(21) Appl. No.: 10/117,416

(22) Filed: Apr. 7, 2002

(51) Int. Cl. A61L 9/20

(52) U.S. Cl. 422/4; 433/82; 422/24; 422/121

(57) ABSTRACT

The present invention provides an Ultraviolet apparatus or system for delivering substantially pure compressed air to desired locations. The invention is particularly useful for delivering substantially pure compressed air to dental unit handpieces and instruments. The invention provides a dental chair unit including apparatus for delivering substantially pure 99.9% compressed air and method for delivering substantially pure compressed air to a dental handpieces, other medical applications and also in industrial automotive assemblies with pneumatic tools.

### TECHNICAL FIELD OF THE INVENTION

The present invention relates to a method where an Ultra-Violet light is housed in a Stainless Steel Chamber with a Quartz Sleeve. As compressed air flows over the UV lamp, the air is instantly disinfected to 99.9%. Clean & Disinfected compressed air is used in Dental, as well as in Medical, Automotive Assembly and any other critical compressed air applications. In one embodiment, the present invention relates to a method and apparatus for the delivery of substantially 99.9% pure compressed air to dental chair units and dental appliances.

### BACKGROUND OF THE INVENTION

In providing dental care to patients, the dental practitioner must always utilize compressed air. Infection control is an ever-present problem in a dental office setting. Dental Compressed Air is used for High-Speed & Low-Speed Handpieces and air syringes. Untreated Compressed air contains tiny particles of dirt, dust, oil, moisture and airborne microbes, which cause premature wear on High-Speed & Low-Speed Handpieces and where moisture supports bacterial growth in supply lines. This contamination travels directly into patients' mouths and to the Dental Team, whose exposure is at least 30 times more than the patient's exposure. This system eliminates one of the hazards associated with the dental workplace. In fact, dental patients having compromised immune systems, such as elderly patients or patients having asthma, cancer, diabetes, HIV and AIDS, are at an increased risk of infection from contaminated dental unit air lines.

In the Dental Unit Air System, Compressed Air is delivered from a reservoir through Copper tubing or piping to dental Handpieces and instruments located at the dental chair. Ambient Temperature Air contains moisture, dust and airborne bacteria which when compressed must be filtered and dried. Furthermore, to filter airborne bacteria is extremely difficult and not cost effective. A low micron filter (0.22 micron) will reduce pressure, frequent interruptions in changes of filter, and to reach the same output, larger Air compressor with increased power & equipment cost would require. (over $1,500 to $2,000) Copper tubing condensates during temperature changes causing moisture, which supports bacteria growth and Copper Oxides (Rust). Copper Oxychloride Synonyms—curpic oxychloride compounds such as “Bluish-Green”powder—Rust, occurs on the outside and inside of the tubing. This bright bluish-green powder is extremely poisonous & toxic both through ingestion and inhalation. Manufacturers use it in Pigment, pesticide and insecticide to kill plant fungus in grapevines.

The American National Standard/American Dental Association Specification No. 94—for Dental Compressed Air Quality, Procedure 5.1.5 Odor Limit states: The compressed air shall not contain objectionable odors. Odor Test Procedure 7.1.9: to sample compressed air, blow into odorless 11.8″×11.8″×11.8″ box for one minute, and then it is immediately smelled by observer. Five independent observers shall each smell independent samples of the air; if more than one finds the samples objectionable in odor, then compressed air system fails this test. No further information is provided on what to do with contaminated air lines. ODOR indicates that microorganism growth is present in the air lines. The Organization of Safety & Asepsis reports that “Both altered nasal flora and serological evidence of exposure to Legionella bacteria have been reported in dental healthcare workers.” They go on to say that “A published case report associated two postoperative Pseudomonas infections in immuno-compromised patients with exposure to contaminated dental coolant water.”¹

This new design & method allows us to take sample of compressed air at the discharge end by using a “PRESTERILIZED” membrane Filter to collect all contaminants and bacteria. The filter is manufactured by Millipore Corporation. The Certified TYPE HA 0.45 um PORE SIZE is used for bacteriological analysis of potable waters in accordance with procedures referenced in Standard Methods (current edition), and laboratory standards for equipment and materials set forth by the U.S. Environmental Protection Agency. All 100% of Pure Compressed air is passed through the sample chamber fitted with the presterilized Filter membrane and then allowed to incubate for 72 hours with 1 ml of Sterile distilled water. Any Bacteria will grow on the membrane as colonies. (CFU's). Most water and air supply lines are in Copper and are installed under concrete floors that make repairs or replacements very expensive and difficult to clean. As the compressed air enters the long tubing of the dental unit (the air lines that feed into the dental Handpieces), the potential for biofilm formation and microorganism growth increases. Air in the dental office, therefore, has the potential to cause infection to both patients and dental staff, due to contamination by microorganisms and the like. During dental treatment using a dental chair unit the high-speed handpieces produce a very fine aerosol mist, and also, the patient's oral cavity is rinsed with water & air that is delivered by a dental instrument such as air-water syringe. Some dental chairs have a self-contained water supply bottle filled with Sterile water, which is pushed by compressed air. The compressed air contaminates the pure sterile water in the self-contained bottles, and allows microorganism growth, and a potential risk for patient, Doctor and assistants.

U.S. Pat. No. 5,526,841 to Detsch, et al. discloses a dental water line decontamination unit comprising a water supply, a disinfectant supply, a source of pressurized gas and a selector valve having separate inlets connected to the water supply, the disinfectant supply and the pressurized gas source.

Dental Handpieces and instruments are typically worn by the use of unfiltered compressed air containing particulate contamination, requiring frequent repair or replacement. Dental handpieces, such as high-speed drills, operate at about 300,000 to 500,000 RPM, and even minute particles in the air will cause friction and erosion of the internal turbine surfaces of the instrument.

To date, no fully automatic method or system exists in the art to deliver uncontaminated compressed air to a dental chair unit. As such, there remains a great need in the art to provide methods and apparatus to eliminate bacteria and biofilm formation in dental unit air lines and to deliver substantially pure, uncontaminated and safe compressed air to a dental chair unit and dental instruments that come into contact with a dental patient.

### SUMMARY OF THE INVENTION

The present invention provides a method and apparatus for the delivery of substantially pure compressed air to dental unit Handpieces and instruments. The present invention further provides a reduction of particulate matter introduced into the compressed air supplied to the dental unit Handpieces and instruments.

These features, together with the advantages thereof the Ultra-Violet Disinfection/Sterilization chamber system is an existing art, which shall become apparent from the specification which follows, are accomplished by the invention as hereinafter described and claimed.

The present invention, therefore, provides a method for delivering substantially pure compressed air to dental unit Handpieces and instruments comprising: an apparatus capable of supplying substantially 99.9% pure compressed air, said apparatus comprising a pressurized disinfection/sterilization Stainless Steel chamber containing UV Lamp radiating at 254 nanometers with power flux of 40,000 microwatts per second per square centimeter, which is 50% higher than needed to kill the most difficult bacteria and viruses in water. Ultra-Violet light is twice as effective in Germicidal air disinfection than in water. UV Chamber is in fluid communication therewith, and corrosion resistant and non-leachable tubing fluidly connecting said pressurized reservoir & UV Chamber, said at least one anti-back flow valve and at least one dental handpiece or instrument; and delivering said substantially pure compressed air to said dental unit handpieces and instruments through said apparatus.

In another embodiment, the apparatus further includes a Extractor/Dryer® Filter means positioned distal to the Pressurized reservoir and in fluid communication with said Ultra-Violet Chamber, said anti-back flow valve and said at least one dental handpiece or instrument.

The invention further provides, in combination with a dental chair, a dental unit including at least one dental handpiece or instrument; and an apparatus for delivering substantially pure compressed air to said at least one dental unit handpiece or instrument, said apparatus comprising a pressurized reservoir, Extractor/Dryer® Filter, UV Chamber for disinfecting compressed air; at least one anti-back flow valve positioned distal from and in fluid communication with said pressurized reservoir, Extractor/Dryer® Filter, UV Chamber and corrosion resistant and non-leachable tubing fluidly connecting said reservoir, Extractor/Dryer® Filter, UV Chamber, said at least one anti-back flow valve and said at least one dental handpiece or instrument, wherein said apparatus delivers substantially pure compressed air to dental unit handpieces or instruments.

### BRIEF DESCRIPTION OF THE DRAWING

FIG. 1 is a schematic representation of one embodiment of the apparatus for the delivery of substantially pure compressed air.

### DETAILED DESCRIPTION OF THE INVENTION

In general, the present invention provides a method and apparatus for the delivery of substantially pure compressed air to medical devices, such as dental unit chairs, dental handpieces, dental instruments and the like. The invention further includes a method for substantially reducing or eliminating the proliferation of microorganisms and biofilm within the lines or tubing of a dental unit air line.

The phrase “substantially pure compressed air” refers to air that is at least about 99.9% free of contamination from microorganisms, including bacteria, viruses, fungi, protozoa, dust, oil and moisture. In one embodiment, compressed air is subjected to filtration and disinfection/sterilization process to obtain the substantially pure compressed air for delivery to the dental handpieces and instruments.

The term “biofilm” refers to a film or layer of microorganism growth that is attached or adhered to the inner walls of the conduits, passages, and tubing of a dental air lines. Due to the pressure of air that flows through the dental air lines, portions of the biofilm may detach from the inner walls and contaminate the dental air & water that is destined to reach to the oral cavity of the dental patient.

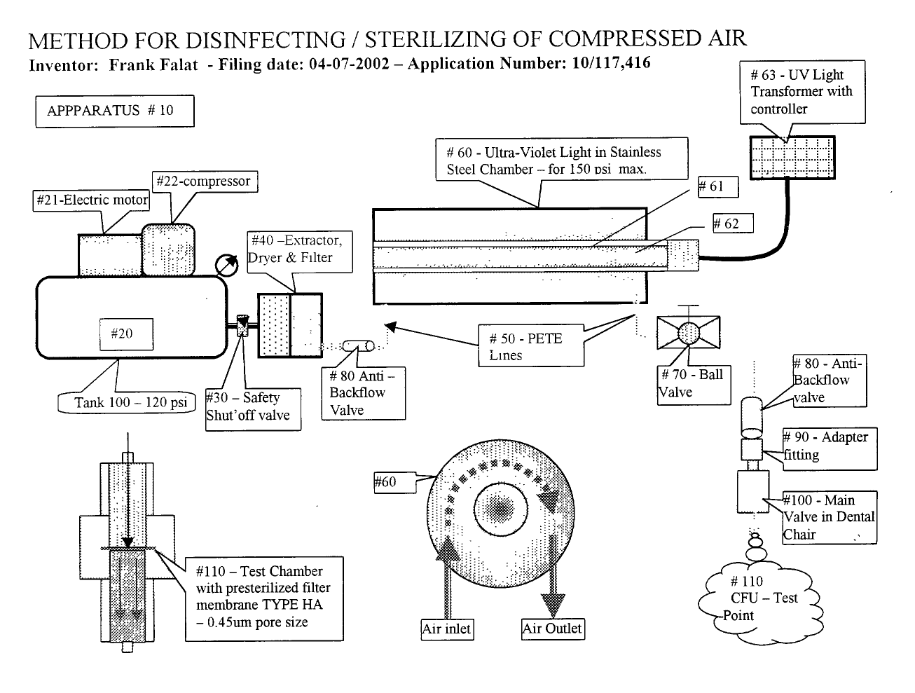

While the apparatus described herein can be used to deliver substantially pure compressed air for dental applications and for a variety of medical or other applications (commercial or industrial), the present invention will be described in greater detail with reference to FIG. 1, which depicts one embodiment of the compressed air delivery apparatus adapted for delivery of substantially pure compressed air to the dental handpieces and instruments of a dental chair unit.

A dental unit air line traditionally comprises flexible tubing or copper tubing connecting an air line supplies to dental handpieces and instruments. The dental handpieces and instruments generally include a nozzle and an automatic or manual control to permit discharge of the dental compressed air from the tubing through the nozzle to dry the oral cavity of the patient during dental treatment.

An apparatus that is capable of delivering substantially pure compressed air to dental unit handpieces and instruments is provided by the present invention. The apparatus includes a pressurized reservoir for storing the compressed air, Extractor/Dryer® Filter, UV Chamber System, at least one anti-back flow valve positioned distal in fluid communication with and corrosion resistant and non-leachable tubing fluidly connecting said pressurized reservoir, Extractor/Dryer® Filter, UV Chamber system and the anti-back flow valve with the dental handpieces or instruments.

The Compressor with pressurized reservoir (tank) contains or stores, under constant pressure, which is delivered to the dental handpieces and instruments. The compressed air is stored under a pressure of at least about 100-psi, preferably but not higher then maximum of 120 psi. Storing the compressed air in the tank, which is under constant pressure, eliminates pressure changes that are very critical for high-speed instruments (drills).

A least one anti-back flow valve is provided to ensure that contamination from a patient's oral cavity, such as contamination from debris, blood, saliva and tissue, is not retracted or drawn back into the air line by negative pressure. The term “anti-back flow” is intended to mean that the valve allows flow of substantially pure compressed air in the direction of the dental handpieces and instruments, but prevents reverse flow back into the tubing of the apparatus. The anti-back flow valve is designed such that as little as 1 psi of pure air pressure against the valve will cause it to open to allow compressed pure air to flow through. When no air pressure, that in any pressure greater than 0-psi pressure, is exerted against the anti-back flow valve, the valve remains in the closed position. While in the closed position, the anti-back flow valve impedes the flow of air back into flexible tubing of the pure compressed air delivery apparatus.

The anti-back flow valve generally includes a body, a spring and an activated poppet or ball. The anti-back flow valve may be constructed from stainless steel or a polymeric material. In a preferred embodiment, the body, spring and poppet/ball of the anti-back flow valve are manufactured from stainless steel. Stainless steel is a general term for a group of steel products that comprise chrome, nickel, molybdenum, titanium and carbon. A preferred stainless that can be used to construct the anti-back flow valve in known in the art as 316 stainless steel. 316 stainless steel is a grade of stainless steel that includes about 18% chromium and is approved for food and medical applications. 316 stainless steel is corrosion resistant and non-leachable.

As described above, the anti-back flow valve can be manufactured from a polymeric material. A suitable polymeric material used to construct the body of the antiback flow valve is polyvinylidene, which is non-corrosive and exhibits excellent chemical resistance. The anti-back flow valve preferably has an O-ring. An especially useful material that is used to construct the O-ring is fluoroelastomer that is commercially available from Dupont Dow Elastomers under trademark VITON®. The spring and ball of the anti-back flow valve can be made of stainless steel.

As described above, the pressurized Compressor Tank, Extractor/Dryer® Filter, UV Disinfecting/Sterilizing Chamber, tubing, piping or lines fluidly connect anti-back flow valve and dental handpieces or instruments. The tubing is flexible, corrosion resistant, and non-leachable. Leaching is a process by which a soluble substance is separated from a solid by washing with water moisture or another substance. The term “non-leachable” refers to the ability of the tubing to resist the leaching process, thereby not releasing any substances from the inner walls of the tubing when air with moisture is continuously flowing through the tubing. The flexible tubing should be approved by the Food and Drug Administration (FDA), National Sanitation Foundation (NSF) and the United States Department of Agriculture for use in food and medical applications. Preferably, the flexible tubing is made from a FDA, NSF and USDA approved polymeric material. Suitable polymeric materials used to manufacture the flexible tubing include, not are limited to, polyalkylenes, such as polyethylene and polypropylene. In a preferred embodiment, the flexible tubing is manufactured from an approved polypropylene material that has been listed with NSF Standards 51 and 61 as non-leachable for use in medical and food applications.

In another embodiment, the apparatus further includes an Extractor/Dryer® Filter means. The Extractor/Dryer® Filter means is positioned distal to the compressor tank, UV Disinfecting/Sterilization Chamber and is in fluid communication with the anti-back flow valve and the dental handpieces and instruments.

The Extractor/Dryer® Filter means preferably includes a cartridge mesh filter material in chamber #1 to capture large particles and causes the formation of larger water droplets. In chamber #2 moisture droplets condense on the honeycomb material and water flows along bottom and out the drain. Last chamber #3 has second stage wire supported fiber filter to capture remaining dust particles down to 5 micron, exhaust air is clean and dry—no dust, rust, oil or water drops to disrupt air equipment operation.

This Extractor/Dryer® filter is commercially available from LA-MAN® Corporation, Florida with several models vary in SCFM. U.S. Pat. No. RE 32989 and Patented in CANADA 1990—#904-304-0411.

In another embodiment, the invention provides an apparatus for delivering substantially pure compressed air to dental unit handpieces and instruments that includes a means for providing substantially pure compressed air, a pressurized reservoir (compressor tank) for storing the pressurized/compressed air, Extractor/Dryer® Filter, UV Lamp with High pressure Chamber, at least one anti-back flow valve positioned distal to the pressurized tank and in fluid communication the reservoir (compressor tank). Corrosion resistant and non-leachable tubing, as described above, fluidly connects the means for providing substantially pure compressed air, the pressurized reservoir, Extractor/Dryer® Filter, UV lamp with high pressure Chamber and the at least one anti-back flow valve with the dental handpieces or instruments. The apparatus delivers substantially pure compressed air to dental unit handpieces and instruments, and the tubing remains substantially free from microorganism growth and biofilm formation.

According to this embodiment, the means for providing substantially pure compressed air is an Ultra-Violet Lamp in a Quartz Sleeve with High Pressure Stainless Steel Chamber. Several Manufacturers offer their proprietary designs which all are designed for water purification and approved by FDA for drinking water. Since water pressure varies in about 40 to 60 or up to 80 psi., most chambers are not built for higher-pressure rating. Our Stainless Steel Chamber is rated for Maximum Operating Pressure of 125 to 150 psi. Atlantic Ultraviolet Corporation, Hauppauge, N.Y., or R-Can Environmental Inc. in Guelph, Ontario, Canada, manufactures UV system which is incorporated herein by reference, discloses ultraviolet water purifier units that can be used in the apparatus of the present invention. UV AIR Solutions, Inc. 218 Jones Drive, Brandon, Vt. 05733 USA manufactures UV technology for Heating, Ventilating, and Air Conditioning Air disinfecting in Residential and Commercial Buildings. It is used widely in industry to keep food and liquids germ-free during processing and packaging. It's also used in aircraft ventilation systems to prevent infection. Ultraviolet technology is one of the best known methods of controlling microorganisms. It controls airborne pathogens the same way sunlight does, in essence simulating nature. UV operates without negative environmental impact—there are no harmful byproducts to the process and it is energy efficient.

The invention provides a method for delivering substantially Pure Compressed Air to dental unit handpieces and instruments. Corrosion resistant and non-leachable tubing fluidly connects the Tank, Filter, UV chamber and the at least one anti-backflow valve to the handpieces or instruments. The Pure Compressed Air is delivered to the dental patient at about 60-75 psi. The entire apparatus including, most importantly, the flexible tubing remains substantially free from contamination from microorganism growth and biofilm formation.

### EXAMPLE 1

One preferred embodiment of the present invention will be described in greater detail with reference to FIG. 1. FIG. 1 depicts an apparatus 10 for delivering substantially Pure Compressed Air. Apparatus 10 includes an air compressor Tank 20 for providing compressed air. In a more preferred embodiment, air compressor has electric motor 21 and piston compressor 22. Holding tank 20 storing air under pressure of 120 psi. until needed. On demand, the compressed air flows from pressurized tank 20 through a Safety Shutoff valve 30 to Extractor/Dryer® Filter 40 and removing dirt, dust, oil and moisture from air and connected with Tubing 50 to UV Stainless Steel Chamber 60. High output UV Lamp 62 is protected from air pressure and contaminants with Quartz Sleeve 61 sealed with O rings. Output of UV Stainless Steel Chamber 60 is in fluid communication with tubing 50 and Ball Valve 70 through the Anti-backflow valve 80 with Reducing Adapter Fitting 90 to the chairside dental utility Main valve 100. Test Sample Chamber 110 allows us to sample pure compressed air for bacteriological analysis by connecting to any outlet or instruments.

The method of delivering substantially Pure Compressed Air provides air that is substantially free from dirt, dust, oil, moisture, viruses, fungi and airborne bacteria. As the method of the invention decreases the risk for contamination of dental air lines and biofilm, it protects the health and safety of the dental patient and dental staff.

Because the method and apparatus of present invention substantially eliminates the presence of air borne bacteria and dirt, dust, oil and moisture normally found in open air, the method will effectively reduce the cost expended by dental practitioner to repair or replace damaged dental instruments, which provides a considerable savings.

High-speed dental handpieces and instruments are designed with very tight clearances between the turbine and the body that houses the turbine to produce high speeds, namely, in the range of about 300,000 to about 500,000 RPM. Even low amounts of contamination from dirty air will erode the turbine and increase the clearance between the turbine and the body of the dental handpieces and instruments. The increase in the clearances between the turbine and the body results in reduces speed, which would be unacceptable for dental procedures.

The apparatus for delivering substantially Pure Compressed Air is a totally closed system that is fully automatic. The method and apparatus prevents the back-flow from dental handpieces, syringes and the like. The Substantially Pure Compressed Air, Tubing, UV chamber are completely protected from human and airborne contamination. As such, pure compressed air is at least 99.9% pure from microorganism can be delivered to a dental chair unit and to dental tools and instruments, such as air/water syringes, scalers and handpieces commonly used in the dental office and patient.

Based on the foregoing disclosure, it is therefore demonstrated that the object of the present invention is accomplished & should be the method of delivering of Pure Compressed Air and apparatus described. While the method and apparatus can be utilized in any application that requires delivery of substantially Pure Compressed Air, the method is particularly well suited for use in the dental office. It should be understood that the selection of specific materials that comprise the tubing and connections of the system be determined by one having ordinary skill in the art without departing from the spirit of the invention herein disclosed and described. It should therefore be appreciated that the present invention is not limited to the specific embodiments described above, but includes variations, modification and equivalent embodiments defined by the following claims.

### References Cited

1. OSAP.org “Dental Unit Waterlines: OSAP Position Paper”, March, 2000; December 2001 http://www.osap.org/issues/pages/water/duwl.htm

Clark A., Bacterial colonization of dental units and the nasal flora of dental personnel. Proc Roy Soc Med 1974; 1974(67):1269

Fotos P. G., Westfall, H H, Snyder I S, Miller R W, Mutchler B M. Prevalence of Legionella-specific IgG and IgM antibody in a dental clinic population. J Hosp Infec 1985;64:1382-5

Reinthaler F F, Mascher F, Stunzner D. Serological examinations for antibodies against Legionella species in dental personnel. J Dent Res 1988:67(6):942-3.

Martin M V, The significance of the bacterial contamination of dental unit water systems. Br.Dent. J 1987;163:152-3.

I claim:

1. A method for delivering substantially pure compressed air to dental unit handpieces and instruments comprising:
Providing an apparatus capable of supplying substantially pure compressed air, said apparatus comprising a Ultraviolet Lamp in Stainless Steel Chamber capable to Disinfect/Sterilize compressed air, pressurized reservoir (tank), Filter and at least one anti-backflow valve positioned distal to said UV Chamber and in fluid communication therewith, and corrosion resistant and non-leachable tubing fluidly connecting said pressurized reservoir (tank), UV Chamber, said at least one anti-back flow valve and at least one dental handpiece or instrument; and delivering said substantially pure compressed air to said dental unit handpieces and instruments through said apparatus.

2. The method of claim 1, wherein said substantially pure compressed air is greater than about 99.9% free of contamination and air borne microbes.

3. The method of claim 1, wherein said at least one anti-back flow valve is opened by an air pressure of about 1 psi.

4. The method of claim 1, wherein said at least one anti-back flow valve is closed when the pressure exerted against the valve is about 0 psi.

5. The method of claim 1, wherein the substantially pure compressed air is prepared by Ultraviolet Lamp in Stainless Steel Chamber is capable to Disinfect/Sterilize and kills air borne microbes.

6. An apparatus for delivering substantially pure compressed air to dental unit handpieces and instruments comprising:
a pressurized reservoir (tank) part of air compressor containing air at 120-psi.;
and Extractor/Dryer® Filter to remove dirt, dust, oil, & moisture;
and Ultraviolet Lamp in Stainless Steel Chamber capable to Disinfect/Sterilize compressed air; at least one anti-backflow valve positioned distal to UV Chamber and in fluid communication therewith; and corrosion resistant and non-leachable tubing fluidly connecting said pressurized reservoir, UV Chamber, said at least one anti-back flow valve and at least one dental handpiece or instrument.

7. The apparatus of claim 6, wherein said pressurized reservoir (tank) maintains a pressure within the system of at least about 100-psi.

8. The apparatus of claim 6, wherein said corrosion resistant and non-leachable tubing is a flexible polymeric material.

9. The apparatus of claim 8, wherein said flexible polymeric material is selected from the group consisting of polyethylene and polypropylene.

10. The apparatus of claim 9, wherein said flexible polymeric material is polyethylene.

11. The apparatus of claim 6, wherein said anti-back flow valve is opened by an air pressure of about 1-psi.

12. The apparatus of claim 6, wherein said at least one anti-back flow valve is closed when the air pressure exerted against the valve is about 0 psi.

13. The apparatus of claim 6, further comprising a Extractor/Dryer® Filter means disposed distal to said pressurized reservoir (tank) and in fluid communication with Ultraviolet Lamp Stainless Steel Chamber, said at least one anti-back flow valve and said at least one dental handpiece or instrument.

14. The apparatus of claim 13, wherein said Extractor/Dryer® Filter means comprises a fiber cartridge in chamber #1 to capture large particles, chamber #2 where moisture droplets condense on honeycomb material and water flows along bottom and out the drain. Chamber #3 has second stage wire supported fiber filter to capture remaining particles down to 5 micron and exhaust air is clean and dry from dust, rust, oil and water drops but not cleaned from air borne microbes.

15. The apparatus of claim 14, wherein said fiber cartridge material

16. The apparatus of claim 14, wherein said second stage water and oil are absorbed and clean & dry air and in fluid communication with UV Chamber; corrosion resistant and non-leaching tubing fluidly connecting substantially delivering pure compressed air, said at least one anti-back flow valve and at least one handpiece or instrument.

17. The apparatus of claim 16, wherein said means for providing substantially pure compressed air is Ultraviolet Lamp operating at 254 nanometers Inside Stainless Steel Chamber, protected with Quartz sleeve for optimum operating temperatures.

18. The apparatus of claim 17, wherein said means Ultraviolet lamp is Germicidal and most effective against airborne microorganisms.

19. The apparatus of claim 17, wherein said is operating at 40,000 microwatt per seconds per square centimeter.(no less than 32,000)

20. The apparatus of claim 17, wherein said life is 10,000 hours and output after 1 year no less than 25,000 microwatts.

21. The apparatus of claim 20, wherein said 50% higher power than need to kill the most difficult bacteria and viruses. Lamp stays on continuously 24 hours and 365 days.

22. The apparatus of claim 17, wherein said is UV Lamp and Chamber is providing substantially pure compressed air is disinfected/sterilized from airborne microbes.

23. The apparatus of claim 17, wherein said is Stainless steel grade 304 or 316 corrosion resistant and non-leachable material approved by FDA.

24. The apparatus of claim 16, wherein said flexible polymeric material is selected from group consisting of polyethylene and polypropylene.

25. The apparatus of claim 16, wherein said flexible polymeric material is polyethylene.

26. The apparatus of claim 16, wherein said anti-back flow valve is opened by an air pressure of about 1-psi.

27. The apparatus of claim 16, wherein said at least one anti-back flow valve is closed when the air pressure exerted against the valve is about 0 psi.

28. The apparatus of claim 16, further comprising a Extractor/Dryer® Filter means disposed distal to said pressurized reservoir (tank) and in fluid communication with said UV Chamber, said at least one anti-back flow valve and said at least one dental handpiece or instrument.

29. A dental chair unit comprising:
A dental chair;
A dental unit including at least one dental handpiece or instrument; and
An apparatus for delivering substantially pure compressed air to said at least one dental unit handpiece or instrument, said apparatus comprising a pressurized reservoir (tank) for storing compressed air; at least one Extractor/Dryer® Filter; one UV Chamber, and one anti-back flow valve positioned distal from and in fluid communication with said UV Chamber; corrosion resistant and non-leaching tubing fluidly connecting said UV Chamber, said at least one anti-back flow valve and said at least one dental handpiece or instrument, wherein said apparatus delivers substantially pure compressed air to dental unit handpieces or instruments.

30. The dental chair unit of claim 29, wherein said apparatus further comprises a means for providing substantially pure compressed air.

31. The dental chair unit of claim 29, wherein said apparatus further comprises a filter means disposed between said reservoir; UV chamber and at least one anti-back flow valve and in fluid communication therewith.

32. A method for delivering substantially pure compressed air to dental unit handpieces and instruments comprising:
Providing an apparatus for delivering substantially pure compressed air, said apparatus comprising means for providing substantially pure compressed air, a pressurized reservoir containing compressed air, Extractor/Dryer® Filter, UV Lamp in Stainless Steel Chamber, at least one anti-back flow valve positioned distal to said pressurized reservoir (tank) and in fluid communication therewith, and corrosion resistant and non-leaching tubing fluidly connecting said means for providing substantially pure compressed air, said at least one anti-back flow valve and at least one dental handpiece or instrument; and delivering said substantially pure compressed air to said dental unit handpieces and instruments through said apparatus.
